(19) United States
(12) Patent Application Publication
(10) Pub. No.: US 2002/0128496 A1
(43) Pub. Date: Sep. 12, 2002

(54) Process for the preparation of matrix metalloproteinase inhibitors

(76) Inventors: Sou-Jeng Chang, Prairie View, IL (US); Dilinie P. Fernando, Gurnee, IL (US); Ashok Gupta, Gurnee, IL (US); David R. Hill, Gurnee, IL (US); Steven J. Wittenberger, Mundelein, IL (US)

Correspondence Address: ABBOTT LABORATORIES, DEPT. 377 - AP6D-2, 100 ABBOTT PARK ROAD, ABBOTT PARK, IL, 60064-6050, US

(21) Appl. No.: 10/044,035

(22) Filed: Jan. 11, 2002

Related U.S. Application Data
(60) Provisional application No. 60/261,626, filed on Jan. 12, 2001.

(51) Int. Cl. C07D 317/44

(52) U.S. Cl. 549/445

(57) ABSTRACT

The present invention discloses a process for the synthesis of reverse hydroxamate matrix metalloproteinase (MMP) inhibitors.

### CROSS-REFERENCE TO RELATED APPLICATIONS

This application claims priority to U.S. Provisional Patent Applications Ser. No. 60/261,626, filed Jan. 12, 2001, which is hereby incorporated by reference in its entirety.

### TECHNICAL FIELD

The present invention is directed to a process for the preparation of matrix metalloproteinase inhibitors and to intermediates useful in the process.

### BACKGROUND OF THE INVENTION

The matrix metalloproteinases (MMP's) are a class of extracellular enzymes including collagenase, stromelysin, and gelatinase which are believed to be involved in the tissue destruction which accompanies a large number of disease states varying from arthritis to cancer.

There has been heightened interest in discovering therapeutic agents which bind to and inhibit MMP's. The discovery of new therapeutic agents possessing this activity will lead to new drugs having a novel mechanism of action for combating disease states involving tissue degenerative processes including, for example, rheumatoid arthritis, osteoarthritis, osteopenias such as osteoporosis, periodontitis, gingivitis, corneal, epidermal or gastric ulceration, and tumor growth and metastasis.

While commonly owned WO 00/44739, filed Jan. 27, 2000, teaches the preparation of reverse hydroxamate-containing MMP inhibitors, the synthesis is not amenable to large-scale preparation. For example, the formylation of the N-hydroxylamine intermediate is accomplished using formic acetic anhydride to provide the formylated product in a modest 42% yield, making the procedure impractical for large-scale synthesis. In addition, the six-step synthesis provides the final product in 25% overall yield, making the process too inefficient for use on a large-scale.

As shown by the above examples, there is still a need in the pharmaceutical manufacturing industry for the efficient preparation of reverse hydroxamate-containing MMP inhibitors. The present invention discloses a synthesis of MMP inhibitors which offers higher overall yields, making it amenable to large-scale synthesis.

### SUMMARY OF THE INVENTION

In one embodiment the present invention discloses a process for preparing a compound of formula (3)

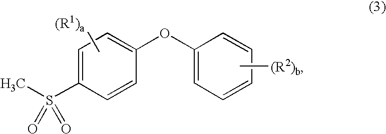

or a therapeutically acceptable salt thereof, wherein

a is 0, 1, or 2;

b is 0, 1,2, or 3;

each R¹ is independently selected from the group consisting of alkyl, halo, nitro, and perfluoroalkyl; and

each R² is independently selected from the group consisting of alkoxy, alkyl, perfluoroalkoxy, and perfluoroalkyl;

the process comprising:

reacting a compound of formula (1)

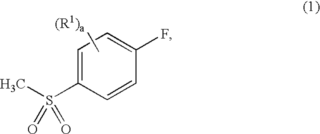

with a compound of formula (2)

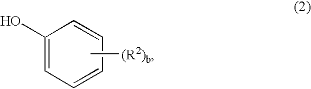

in the presence of a base.

In a preferred embodiment the present invention discloses a process for preparing a compound of formula (3) wherein

a is 0;

b is 1; and

R² is perfluoroalkoxy.

In a more preferred embodiment the compound of formula (3) is 1-(methylsulfonyl)-4-[4′-(trifluoromethoxy)phenoxy]benzene.

In another embodiment the present invention discloses a process for preparing a compound of formula (3),

the process comprising:

(a) reacting a compound of formula (4)

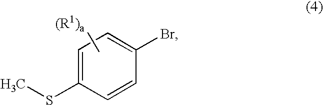

wherein a and R¹ are as previously defined;

with an oxidizing agent; and

(b) reacting the product of step (a) with a compound of formula (2) in the presence of a base.

In another embodiment the present invention discloses a process for preparing a compound of formula (8)

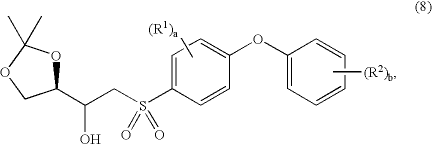

or a therapeutically acceptable salt thereof, wherein

a, b, R¹, and R² are as previously defined;

the process comprising:

(a) reacting a compound of formula (3) with a base;

(b) reacting the product of step (a) with a compound of formula (6)

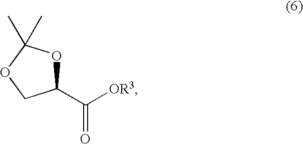

wherein R³ is alkyl; and

(c) reacting the product of step (b) with a reducing agent.

In a preferred embodiment the present invention discloses a process for preparing a compound of formula (8) wherein

a is 0;

b is 1; and

R² is perfluoroalkoxy.

In another embodiment the present invention discloses a process for preparing a compound of formula (10)

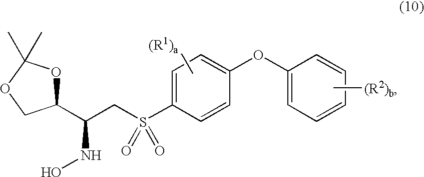

or a therapeutically acceptable salt thereof, wherein

a, b, R¹, and R² are as previously described;

the process comprising:

(a) reacting a compound of formula (8) with methanesulfonyl chloride in the presence of a base; and

(b) reacting the product of step (a) with N-hydroxylamine.

In a preferred embodiment the present invention discloses a process for preparing a compound of formula (10) wherein

a is 0;

b is 1; and

R² is perfluoroalkoxy.

In a more preferred embodiment compound of formula (10) is (4S)-4-[(1S)-1-(hydroxyamino)-2-({4-[4′-(trifluoromethoxy)phenoxy]phenyl}sulfonyl)ethyl]-2,2-dimethyl-1,3-dioxolane.

In another embodiment the present invention discloses a process for preparing a compound of formula (11a)

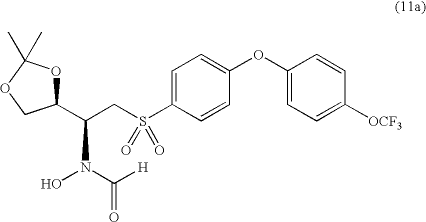

the process comprising:

(a) reacting a compound of formula (1a)

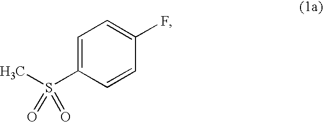

with a compound of formula (2a)

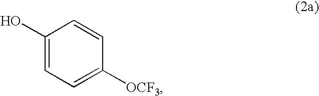

in the presence of a base;

(b) reacting the product of step (a) with a base;

(c) reacting the product of step (b) with a compound of formula (6a)

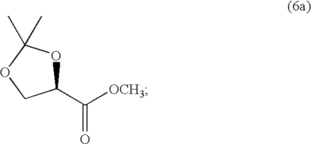

(d) reacting the product of step (c) with a reducing agent;

(e) reacting the product of step (d) with methanesulfonyl chloride in the presence of a base;

(f) reacting the product of step (e) with N-hydroxylamine; and

(g) reacting the product of step (f) with a formylating agent.

In another embodiment the present invention discloses a process for preparing a compound of formula (11a),

the process comprising:

(a) reacting a compound of formula (1a) with a compound of formula (2a) in the presence of potassium hydroxide in dimethyl sulfoxide at about 75° C. to about 100° C. for about 8 to about 24 hours;

(b) reacting the product of step (a) with a mixture of n-butyllithium and lithium hexamethyldisilazide in a mixture of tetrahydrofuran and hexanes at about −78° C. to about −40° C. for about 1 to about 6 hours;

(c) reacting the product of step (b) with a compound of formula (6a) in a mixture of tetrahydrofuran and hexanes at about −78° C. to about −40° C. for about 30 minutes to about 6 hours;

(d) reacting the product of step (c) with sodium borohydride in a mixture of ethanol and tetrahydrofuran at about −20° C. to about 5° C. for about 30 minutes to about 12 hours;

(e) reacting the product of step (d) with methanesulfonyl chloride in the presence of triethylamine in ethyl acetate at about −10° C. to about 30° C. for about 1 to about 8 hours;

(f) reacting the product of step (e) with N-hydroxylamine in a mixture of water and methyl tert-butyl ether at about −20° C. to about 0° C. for about 4 to about 24 hours; and

(g) reacting the product of step (f) with 2,2,2-trifluoroethyl formate and formic acid in buffered isopropyl acetate at about 45° C. to about 75° C. for about 1 to about 10 hours.

In another embodiment the present invention discloses a process for preparing a compound of formula (11a),

the process comprising:

(a) reacting a compound of formula (4a)

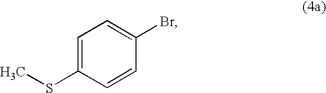

with an oxidizing agent;

(b) reacting the product of step (a) with a compound of formula (2a) in the presence of a base;

(c) reacting the product of step (b) with a base;

(d) reacting the product of step (c) with a compound of formula (6a);

(e) reacting the product of step (d) with a reducing agent;

(f) reacting the product of step (e) with methanesulfonyl chloride in the presence of a base;

(g) reacting the product of step (f) with N-hydroxylamine; and

(h) reacting the product of step (g) with a formylating agent.

In another embodiment the present invention discloses a process for preparing a compound of formula (11a),

the process comprising:

(a) reacting a compound of formula (4a) with potassium peroxymonosulfate in a mixture of ethanol and water at about 20° C. to about 45° C. for about 1 to about 10 hours;

(b) reacting the product of step (a) with a compound of formula (2a) in the presence of potassium phosphate in N,N-dimethylformamide at about 100° C. to about 140° C. for about 8 to about 20 hours;

(c) reacting the product of step (b) with a mixture of n-butyllithium and lithium hexamethyldisilazide in a mixture of tetrahydrofuran and hexanes at about −78° C. to about −40° C. for about 1 to about 6 hours;

(d) reacting the product of step (c) with a compound of formula (6a); in a mixture of tetrahydrofuran and hexanes at about −78° C. to about −40° C. for about 30 minutes to about 6 hours;

(e) reacting the product of step (d) with sodium borohydride in a mixture of ethanol and tetrahydrofuran at about −20° C. to about 5° C. for about 30 minutes to about 12 hours;

(f) reacting the product of step (e) with methanesulfonyl chloride in the presence of triethylamine in ethyl acetate at about −10° C. to about 30° C. for about 1 to about 8 hours;

(g) reacting the product of step (f) with N-hydroxylamine in a mixture of water and methyl tert-butyl ether at about −20° C. to about 0° C. for about 4 to about 24 hours; and

(h) reacting the product of step (g) with 2,2,2-trifluoroethyl formate and formic acid in buffered isopropyl acetate at about 45° C. to about 75° C. for about 1 to about 10 hours.

### DETAILED DESCRIPTION OF THE INVENTION

The present invention is directed to processes for the preparation of matrix metalloproteinase inhibitors and to intermediates which are useful in these processes of preparation. As used in the specification the following terms have the meanings specified:

As used herein, the singular forms “a”, “an”, and “the” include plural reference unless the context clearly dictates otherwise.

The term “alkoxy,” as used herein, represents an alkyl group attached to the parent molecular moiety through an oxygen atom.

The term “alkyl,” as used herein, represents a monovalent group derived from a straight or branched chain saturated hydrocarbon by the removal of a single hydrogen atom.

The term “base,” as used herein, represents a reagent capable of accepting protons during the course of a reaction. Examples of bases include carbonate salts such as potassium carbonate, potassium bicarbonate, sodium carbonate, sodium bicarbonate, and cesium carbonate; halides such as cesium fluoride; phosphates such as potassium phosphate, potassium dihydrogen phosphate, and potassium hydrogen phosphate; hydroxides such as lithium hydroxide, sodium hydroxide, and potassium hydroxide; alkoxides such as sodium tert-butoxide, potassium tert-butoxide, and lithium tert-butoxide; alkyllithiums such as tert-butyllithium, n-butyllithium, and methyllithium; dialkylamides such as lithium diisopropylamide; disilylamides such as lithium hexamethyldisilazide, potassium hexamethyldisilazide, and sodium hexamethyldisilazide; alkylamines such as triethylamine, diisopropylamine, and diisopropylethylamine; heterocyclic amines such as 4-dimethylaminopyridine, 2,6-lutidine, 1-methylimidazole, pyridine, pyridazine, pyrimidine, and pyrazine; bicyclic amines such as 1,8-diazabicyclo[4.3.0]undec-7-ene; and hydrides such as lithium hydride, sodium hydride, and potassium hydride. The base chosen for a particular conversion depends on the nature of the starting materials, the solvent or solvents in which the reaction is conducted, and the temperature at which the reaction is conducted.

The term “buffered solvent,” as used herein, represents a solvent containing an agent capable in solution of neutralizing acids and bases and thereby maintaining a pH at or near the original pH of a solution during the course of a reaction. Representative buffering agents include sodium formate, sodium acetate, sodium hydrogenphosphate, and the like.

The term “formyl,” as used herein, represents —CHO.

The term “formylating agent,” as used herein, represents a reagent capable of donating a formyl group to the nitrogen atom of a molecule during the course of a reaction. Examples of formylating agents include formic acid; 2,2,2-trifluoroethyl formate; a mixture of formic acid and acetic anhydride; acetic formic anhydride; 2,3,4,5,6-pentafluorophenyl formate; ethyl formate; propyl formate; phenyl formate; and mixtures thereof.

The term “halo,” as used herein, represents F, Cl, Br, or I.

The term “nitro,” as used herein, represents —NO₂.

The term “oxidizing agent,” as used herein, represents a reagent capable of converting a thioether to a sulfone. Preferred oxidizing agents for the practice of the present invention include hydrogen peroxide, potassium peroxymonosulfate (OXONE®), sodium periodate, and potassium permanganate.

The term “perfluoroalkoxy,” as used herein, represents a perfluoroalkyl group attached to the parent molecular moiety through an oxygen atom.

The term “perfluoroalkyl,” as used herein, represents an alkyl group wherein each hydrogen radical bound to the alkyl group has been replaced by a fluoride radical.

The term “reducing agent,” as used herein, represents a reagent capable of converting a ketone to an alcohol. Preferred reducing agents for the practice of the present invention include sodium borohydride, sodium cyanoborohydride, sodium triacetoxyborohydride, and lithium borohydride.

The compounds of the present invention can exist as therapeutically acceptable salts. The term “therapeutically acceptable salt,” as used herein, represents salts or zwitterions of the compounds which are water or oil-soluble or dispersible; suitable for treatment of diseases without undue toxicity, irritation, and allergic response; commensurate with a reasonable benefit/risk ratio; and effective for their intended use. The salts can be prepared during the final isolation and purification of the compounds or separately by reacting the amino group of the compounds with a suitable acid. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isothionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetic, trifluoroacetic, glutamate, para-toluenesulfonate, undecanoate, hydrochloric, hydrobromic, sulfuric, phosphoric, and the like. The amino groups of the compounds can also be quaternized with alkyl chlorides, bromides, and iodides such as methyl, ethyl, propyl, isopropyl, butyl, lauryl, myristyl, stearyl, and the like.

All of the processes of the present invention can be conducted as continuous processes. The term “continuous process,” as used herein, represents steps conducted without isolation of the intermediates.

Because asymmetric centers exist in the present compounds, the invention contemplates stereoisomers and mixtures thereof. Individual stereoisomers of compounds are prepared by synthesis from starting materials containing the chiral centers or by preparation of mixtures of enantiomeric products followed by separation such as conversion to a mixture of diastereomers followed by separation or recrystallization, chromatographic techniques, or direct separation of the enantiomers on chiral chromatographic columns. Starting compounds of particular stereochemistry are either commercially available or are made by the methods described below and resolved by techniques well-known in the art.

Because carbon-carbon double bonds exist in the present compounds, the invention contemplates various geometric isomers and mixtures thereof resulting from the arrangement of substituents around these carbon-carbon double bonds. These substituents are designated as being in the E or Z configuration wherein the term “E” represents higher order substituents on opposite sides of the carbon-carbon double bond, and the term “Z” represents higher order substituents on the same side of the carbon-carbon double bond.

Synthetic Processes

Abbreviations used in the descriptions of the schemes and the examples are as follows: DMSO for dimethyl sulfoxide, DME for 1,2-dimethoxyethane, DMF for N,N-dimethylformamide, NMP for N-methylpyrrolidinone, THF for tetrahydrofuran, LiHMDS for lithium hexamethyldisilazide, and MTBE for methyl tert-butyl ether, and min for minutes.

The methods of this invention will be better understood in connection with the following synthetic schemes which illustrate an embodiment of this invention. It will be readily apparent to one of ordinary skill in the art that the compounds of this invention can be prepared by substitution of the appropriate reactants and agents in the synthesis shown below. Starting materials can be obtained from commercial sources or prepared by well-established literature methods known to those of ordinary skill in the art. The groups R¹, R², R³, a, and b are as previously defined unless otherwise specified.

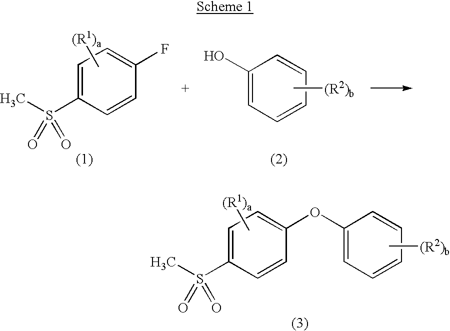

As shown in Scheme 1, compounds of formula (1) can be reacted with compounds of formula (2) in the presence of a base to provide compounds of formula (3). Representative bases include sodium hydroxide, potassium hydroxide, lithium hydroxide, sodium tert-butoxide, potassium tert-butoxide, and lithium tert-butoxide. Examples of solvents used in these reactions include DMSO, DME, water, and dioxane. The reaction is conducted at about 75° C. to 100° C. for about 8 to about 24 hours. In a preferred embodiment, compounds of formula (1) are reacted with compounds of formula (2) in DMSO in the presence of potassium hydroxide at 90° C. for about 10 hours to provide compounds of formula (3).

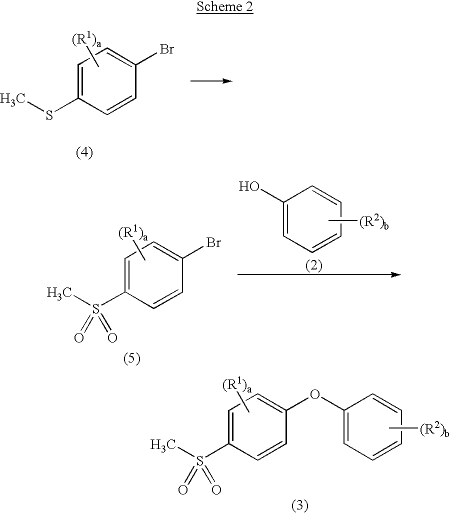

An alternative route to compounds of formula (3) is shown in Scheme 2. Compounds of formula (4) can be treated with an oxidizing agent to provide compounds of formula (5). Representative oxidizing agents include hydrogen peroxide, potassium peroxymonosulfate, sodium periodate, and potassium permanganate. Examples of solvents used in these reactions include ethanol, methanol, water, isopropanol, and mixtures thereof. The reaction is conducted at about 20° C. to about 45° C. for about 1 to about 10 hours. In a preferred embodiment, compounds of formula (4) in a mixture of ethanol and water are reacted with potassium peroxymonosulfate at about 35° C. for about 6 hours to provide compounds of formula (5).

Compounds of formula (5) can be reacted with compounds of formula (2) in the presence of a base to provide compounds of formula (3). Representative bases include potassium phosphate, potassium carbonate, and potassium hydroxide. Examples of solvents used in these reactions include DMF, DMSO, DME, and NMP. The reaction is conducted at about 100° C. to about 140° C. for about 8 to about 20 hours. In a preferred embodiment, compounds of formula (5) are reacted with compounds of formula (2) in DMF in the presence of potassium phosphate at 130° C. for about 13 hours to provide compounds of formula (3).

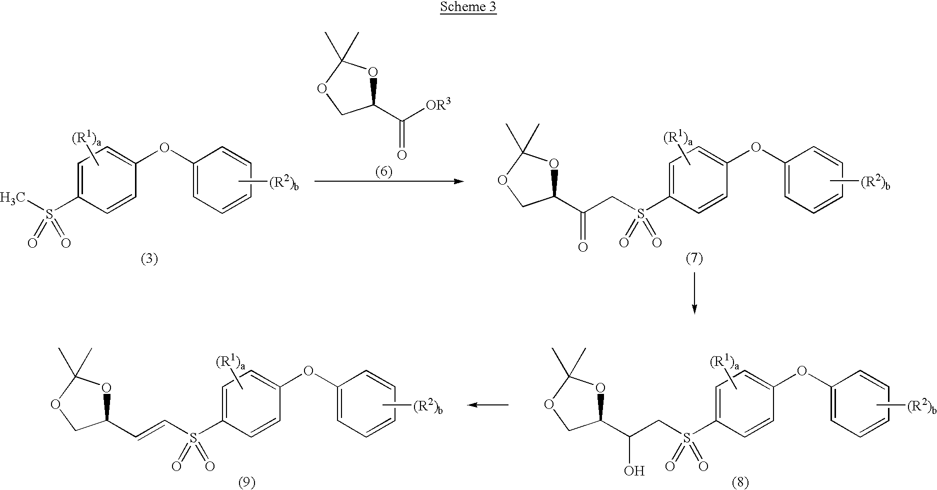

As shown in Scheme 3, compounds of formula (3) can be treated sequentially with base and with compounds of formula (6) to provide compounds of formula (7). Representative bases include n-butyllithium, tert-butyllithium, methyllithium, lithium diisopropylamide, potassium tert-butoxide, lithium hexamethyldisilazide, potassium hexamethyldisilazide, sodium hexamethyldisilazide, and mixtures thereof. Examples of solvents used in these reactions include THF, diethyl ether, MTBE, hexanes, toluene, tetramethylethylenediamine, and mixtures thereof. The reaction is conducted at about −78° C. to about −40° C. for about 2 to about 12 hours. In a preferred embodiment, compounds of formula (3) in THF at −40° C. are treated with lithium hexamethyldisilazide and n-butyllithium in hexanes, stirred for 2 hours, treated with compounds of formula (6), and stirred for 1 hour to provide compounds of formula (7).

Conversion of compounds of formula (7) to compounds of formula (8) can be accomplished by treatment with a reducing agent. Representative reducing agents include sodium borohydride, sodium triacetoxyborohydride, sodium cyanoborohydride, and lithium borohydride. Examples of solvents used in these reactions include ethanol, methanol, THF, diethyl ether, toluene, and mixtures thereof. The reaction is conducted at about −20° C. to about 5° C. for about 30 minutes to about 12 hours. In a preferred embodiment, a suspension of sodium borohydride in ethanol at −5° C. is treated with compounds of formula (7) (either neat or as a solution in THF) and stirred for about 1 hour to provide compounds of formula (8).

Alternatively, compounds of formula (8) can be prepared by treating compounds of formula (3) sequentially with a base and with the appropriate aldehyde (a compound of formula (6) wherein the OR³ is replaced with hydrogen), as described in commonly owned U.S. patent application Ser. No. 09/239,087.

Compounds of formula (8) can be treated with methanesulfonyl chloride in the presence of a base to provide compounds of formula (9). Representative bases include triethylamine, 1,8-diazabicyclo[4.3.0]undec-7-ene, pyridine, 2,6-lutidine, 1-methylimidazole, 4-dimethylaminopyridine, and diisopropylethylamine. Examples of solvents used in these reactions include ethyl acetate, isopropyl acetate, THF, diethyl ether, toluene, and mixtures thereof. The reaction is conducted at about −10° C. to about 30° C. for about 1 to about 8 hours. In a preferred embodiment, compounds of formula (8) in ethyl acetate are treated with triethylamine, cooled to −5° C., treated with methanesulfonyl chloride, stirred for 1 hour, and warmed to room temperature for about 4 to about 8 hours to provide compounds of formula (9).

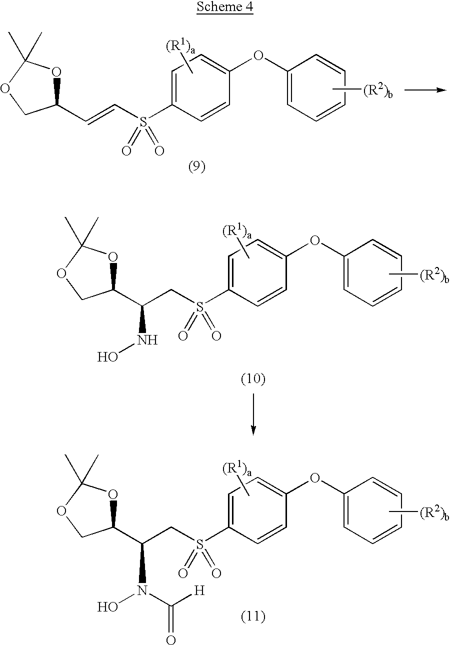

As shown in Scheme 4, compounds of formula (9) can be treated with hydroxylamine to provide compounds of formula (10). Examples of solvents used in this reaction include isopropanol, acetonitrile, ethyl acetate, isopropyl acetate, MTBE, diethyl ether, THF, water, and mixtures thereof. The reaction is conducted at about −20° C. to about 0° C. for about 4 to about 24 hours. In a preferred embodiment, compounds of formula (9) in MTBE at −15° C. are treated with aqueous hydroxylamine and stirred for about 7 to about 20 hours to provide compounds of formula (10).

Conversion of compounds of formula (10) to compounds of formula (11) can be accomplished by treatment with a formylating agent. Representative formylating agents include 2,2,2-trifluoroethyl formate, ethyl formate, propyl formate, and phenyl formate. Examples of solvents used in these reactions include isopropyl acetate, MTBE, THF, ethyl acetate, and n-propyl acetate, all of which can be optionally buffered. The reaction is conducted at about 45° C. to about 75° C. for about 1 to about 10 hours. In a preferred embodiment, compounds of formula (10) in isopropyl acetate buffered with sodium formate and formic acid at 60° C. are reacted with 2,2,2-trifluoroethyl formate for about 5 hours to provide compounds of formula (11).

The invention will now be described in connection with other particularly preferred embodiments of Schemes 1-4, which are not intended to limit its scope. On the contrary, the invention covers all alternatives, modifications, and equivalents which are included within the scope of the claims. Thus, the following examples will illustrate an especially preferred practice of the invention, it being understood that the examples are for the purposes of illustration of certain preferred embodiments and are presented to provide what is believed to be the most useful and readily understood description of its procedures and conceptual aspects.

### EXAMPLE 1

### 1-(methylsulfonyl)-4-[4′-(trifluoromethoxy)phenoxy]benzene

A solution of 1-fluoro-4-(methylsulfonyl)benzene (2.2 kg), KOH (906.3 g), 4-(trifluoromethoxy)phenol (2.364 kg) and DMSO (4.4 L) was heated to 90° C. and stirred until HPLC showed <0.5% starting material remained (about 10 hours). HPLC conditions: Zorbax SB-C8 4.6 mm×25 cm; mobile phase was a gradient of 70% water with 0.1% H₃PO₄/30% acetonitrile to 10% water with 0.1% H₃PO₄/90% acetonitrile over 15 minutes at a flow rate of 1.5 mL/min, followed by a five minute hold at 10/90; UV detection at 220 nM. Retention times: starting sulfone, 4.5 min; desired product, 7.8 min.

The reaction mixture was cooled to room temperature, diluted with water (8.8 kg), and extracted with two portions of toluene (24 L and 4.7 L). The combined extracts were washed with 1N NaOH solution (11 kg) and water (2×11 kg), filtered, concentrated to a volume of approximately 6 L, treated with heptane (22 L) with agitation, stirred for 2 hours, and cooled to 0-5° C. until the mother liquor was assayed for the desired product at <5 mg/ML. The precipitate was filtered, washed with heptane (6.6 L) and dried under vacuum (100 mm Hg with nitrogen sweep) at 40° C. to provide 2.0 kg (96.4% wt potency, 89.6% yield) of the desired product. Recrystallization from methanol/water (4:8 v/v) gave the purified product with 98% recovery.

¹H NMR (300 MHz, CDCl₃) δ 7.9 (d, 2H), 7.3 (br d, 2H), 7.1 (d, 4H), 3.1 (s, 3H).

### EXAMPLE 2

### 1-[(4R)-2,2-dimethyl-1,3-dioxolan-4-yl]-2-({4-4′-(trifluoromethoxy)phenoxy]phenyl }sulfonyl)ethanone

A solution of Example 1 (3.327 kg, 98.7% potency, 9.88 mol) in THF (23 L, pre-dried with 3Å molecular sieves) in a flask equipped with an overhead stirrer, an addition funnel, a temperature probe, and a nitrogen inlet was cooled to <−40° C. and treated with 1M LiHMDS in THF (10.08 L, 10.08 mmol) at such a rate as to keep the internal temperature <−40° C. The solution was treated with 2.28M n-butyllithium in hexanes (2.275 L, 5.187 mol), treated with 2.42M n-butyllithium (2.143 L, 5.187 mol) at such a rate as to keep the internal temperature <−40° C., and stirred for 2 hours. The solution was treated with a solution of (R)-methyl-O-isopropylidene glycerate (1.77 kg, 11.066 mol, 1.12 equivalents) in THF (1.77 kg) at such a rate as to keep the internal temperature <−40° C. The resulting mixture was stirred until <1% starting material was observed by HPLC (about 1 hour). HPLC conditions: Zorbax SB-C8 4.6 mm×25 cm column; mobile phase was a gradient of 70% water with 0.1% H₃PO₄/30% acetonitrile to 10% water with 0.1% H₃PO₄/90% acetonitrile over 15 minutes at a flow rate of 1.5 mL/min; followed by 5 minute hold at 10/90; UV detection at 210 nM. Retention times: starting material, 7.8 min; desired product, 15.2 min.

The mixture was warmed to −25° C. and the reaction was adjusted to pH 5.5 with 2N H₂SO₄ (a pH range between 4-6 was optimal to avoid cleavage of the acetonide group and racemization). The internal temperature of the reaction mixture was allowed to rise to between 0° C. and 5° C. during the acid addition giving a clear biphasic solution and allowing accurate measurement of the pH via a pH meter. The solution was treated with isopropyl acetate (33.27 L), stirred, and allowed to settle. The organic phase was washed sequentially with water (14.48 L), 5% NaHCO₃ solution (14.65 kg), and 15% NaCl solution (14.50 kg), and azeotropically distilled with THF until <10% isopropyl acetate remained as determined by gas chromatography. GC-FID conditions: Stabilwax-DB column (Restek Corp. cat#10823, lot#15531A, L=30 m, ID=0.25 mm), heater at 250° C., oven temperature gradient: 40° C. for 0 to 4 min then 10° C./min to 100° C., then hold at 100° C. 10 min, post-run 5 min; 1 μL injection volume. Peak identification: THF, 4.12 min; isopropyl acetate, 4.34 min.

The solution was filtered and concentrated to a weight of approximately 8 kg to provide a solution of the desired product which was used without further purification. However, the final product could be purified by crystallization from isopropyl acetate to provide a white crystalline solid.

¹H NMR (300 MHz, CDCl₃) δ 7.93-7.85 (m, 2H), 7.33-7.25 (m, 2H), 7.20-7.05 (m, 4H), 4.62 (d, 1H), 4.58-4.52 (dd, 1H), 4.30 (d, 1H), 4.22-4.09 (m, 2H), 1.46 (s, 3H), 1.38 (s, 3H).

### EXAMPLE 3

### 1-[(4R)-2,2-dimethyl-1,3-dioxolan-4-yl]-2-({4-[4′-(trifluoromethoxy)phenoxy]phenyl}sulfonyl)ethanol

A mixture of NaBH₄ (240 g) and ethanol (9.8 L) at −5° C. was treated with Example 2 (either isolated or as a THF solution) (4.53 kg, 10.53 mol by assay) and stirred until HPLC showed none of the starting ketone remaining. HPLC conditions: Zorbax SB-C8 4.6 mm×25 cm, mobile phase was a gradient of 70% water with 0.1% H₃PO₄/30% acetonitrile to 10% water with 0.1% H₃PO₄/90% acetonitrile over 15 minutes at a flow rate of 1.5 mL/min; followed by 5 minute hold at 10/90; UV detection at 220 nM. Retention times: starting material, 15 min; desired products (2 diastereomers), 7.8 and 7.9 min.

The mixture was quenched with 2N acetic acid at such a rate as to keep the internal temperature <30° C., concentrated under vacuum at <40° C. to a volume of approximately 9.8 L, and dissolved in ethyl acetate (49 L). The mixture was washed with water (24.5 L) and 15% wt NaCl solution (24.5 L), concentrated to a volume of approximately 9.8 L, azeotropically distilled with ethyl acetate (49 L) to a final volume of approximately 9.8 L, and dissolved in ethyl acetate (44 L) to provide a solution of the desired product which was used directly in the next step.

1H NMR (300 MHz, CDCl₃) δ 7.9 (d, 2H), 7.3 (br d, 2H), 7.1 (m, 4H), 4.1-3.9 (m, 4H), 3.55 (dd, 1H), 3.4-3.1 (m, 3H), 1.43, 1.35, 1.30, 1.23 (s, s, s, s, total of 6H from 2 diastereomers).

### EXAMPLE 4

### (4S)-2,2-dimethyl-4-[(E)-2-({4-[4′-(trifluoromethoxy)phenoxy]phenyl}sulfonyl)ethenyl]-1,3-dioxolane

A solution of Example 3 in ethyl acetate (5.00 kg, 10.53 mol theoretical) and triethylamine (4.32 kg) was cooled to −5° C., treated with methanesulfonyl chloride (1.94 kg) at such a rate as to maintain the internal reaction temperature at <10° C., stirred at 0-5° C. for 1 hour, and then warmed to room temperature until HPLC showed no more than 0.5% starting material or mesylate intermediate (about 4-8 hours). HPLC conditions: Zorbax SB-C8 4.6 mm×25 cm, mobile phase was a gradient of 70% water with 0.1% H₃PO₄/30% acetonitrile to 10% water with 0.1% H₃PO₄/90% acetonitrile over 15 minutes at a flow rate of 1.5 mL/min; followed by 5 minute hold at 10/90; UV detection at 220 nM. Retention times: starting material, 7.8 and 7.9 min; mesylate intermediate, 15.5 min; product, trans vinyl sulfone, 16.0 min; cis vinyl sulfone, 17.1 min. Typical trans/cis ratio is 10:1.

The reaction was quenched with water (14.6 kg) and the organic layer was washed with 10% wt citric acid solution (19.6 kg), followed successively by 10% wt NaHCO₃ solution (19.6 kg) and water (19.6 kg). The organic layer was concentrated to a volume of approximately 9.8 L, azeotropically distilled with MTBE (2×49L), and concentrated to a final volume of approximately 9.8 L. The residue was dissolved in MTBE (49 L), and assayed for residual ethyl acetate by gas chromatography. If ethyl acetate was <5% in area, additional MTBE (25 L) was added to provide the desired product as a solution. If ethyl acetate was >5% in area, an additional azeotropic distillation with MTBE was performed.

1H NMR (300 MHz, CDCl₃) δ 7.1 (m, 4H), 6.9 (dd, 1H), 6.65 (dd, 1H), 4.7 (m, 1H), 4.2 (dd, 1H), 3.7 (dd, 1H), 1.43 (s, 3H), 1.4 (s, 3H).

### EXAMPLE 5

### (4S)-4-[(1S)-1-(hydroxyamino)-2-({4-[4′-(trifluoromethoxy)phenoxy]phenyl}sulfonyl)ethyl]-2,2-dimethyl-1,3-dioxolane

A solution of Example 4 in MTBE was cooled to −15° C., treated with 50% wt aqueous NH₂OH over a period of 30 minutes at such a rate as to keep the internal temperature between −10° C. and −15° C., and stirred until HPLC showed <0.5% starting material (about 7 to 20 hours). HPLC conditions: Zorbax SB-C8 4.6 mm×25 cm, mobile phase was a gradient of 70% water with 0.1% H₃PO₄/30% acetonitrile to 10% water with 0.1% H₃PO₄/90% acetonitrile over 15 minutes at a flow rate of 1.5 mL/min; followed by 5 minute hold at 10/90; UV detection at 220 nM. Retention times: trans vinyl sulfone, 16.0 min; cis vinyl sulfone, 17.1 min; product (syn), 7.6 min; product (anti), 8.0 min.

The mixture was warmed to room temperature, and the organic layer was concentrated to a volume of approximately 9.8 L while maintaining a temperature of <30° C. The residue was dissolved in ethyl acetate (74 L), washed with 15% wt NaCl solution (2×19.6 L) and concentrated to a volume of approximately 9.8 L. The mixture was azeotropically distilled with MTBE (2×49 L) to a final volume of 9.8 L with <10% ethyl acetate relative to MTBE. The concentration of product in solution was adjusted to 40-45% by weight by the removal or addition of MTBE, heptane (14.7 L) was slowly added, and the resulting slurry was stirred for at least 4 hours until the concentration of product in the mother liquor was <30 mg/mL. The precipitate was filtered, washed with cold MTBE/heptane (1:3 v/v, 9.8 L), and dried under vacuum (100 mmHg with nitrogen sweep) at 30° C. to provide 4.82 kg (63.6%) of the desired product with 0.74% of the anti diastereomer.

¹H NMR (300 MHz, CDCl₃) δ 7.9 (d, 2H), 7.3 (d, 2H), 7.1 (br d, 4H), 4.35 (m, 1H), 4.05 (dd, 1H), 3.8 (dd, 1H), 3.6 (m, 1H), 3.45 (m, 1H), 3.1 (dd, 1H), 1.4 (s, 3H), 1.35 (s, 3H).

### EXAMPLE 6

### (1S)-1-[(4S)-2,2-dimethyl-1,3-dioxolan-4-yl]-2-({4-[4′-(trifluoromethoxy)phenoxy]phenyl}sulfonyl)ethyl(hydroxy)formamide

A 100 L flask equipped with an overhead stirrer, a nitrogen inlet, a reflux condenser, and a thermocouple was charged with Example 5 (3.5 kg), sodium formate (0.350 kg), isopropyl acetate (30.45 kg), 2,2,2-trifluoroethyl formate (9.50 kg), and formic acid (1.05 kg). The mixture was heated to an internal temperature 60° C. and maintained at this temperature with continuous stirring until HPLC showed less than 0.5% starting material (about 5 hours). HPLC conditions: Luna C-8 Phenomenex column at 20° C., mobile phase was a gradient of 55% KH₂PO₄ buffer (pH 2.3)/45% acetonitrile to 33/67 over 55 min at a flow rate of 1 mL/min; UV detection at 210 nM. Retention times: starting material, 41.4, product, 32.3 min.

The reaction was cooled to <30° C. and treated with 5% wt sodium chloride solution (17.68 kg). The organic phase was washed with 5% wt sodium bicarbonate solution (17.79 kg portions) until the pH of aqueous layer was ≧8.0, washed with 5% wt sodium chloride solution (17.68 kg) (aqueous phase pH 7.0), stored at ambient temperature for two days, and then combined with product obtained from a second formylation reaction (3.27 kg) to provide approximately 6.60 kg of combined product. The solutions were combined and distilled under vacuum. Residual 2,2,2-trifluoroethanol was removed by azeotropic distillation with isopropyl acetate and monitored by gas chromatography until the ratio of isopropyl acetate to 2,2,2-trifluoroethanol was 1000:1. GC-FID conditions: Stabilwax-DB column (Restek Corp. cat#10823, lot#15531A, L=30m, ID=0.25 mm), heater at 250° C., oven temperature gradient: 40° C. from 0 to 4 min then 10° C./min to 100° C., then held at 100° C. 10 min, post-run 5 min; 1 μL injection volume. Retention times: isopropyl acetate, 4.5 min, 2,2,2-trifluoroethanol, 9.5 min.

The concentration of the solution was adjusted by solvent removal under vacuum to 25% wt product in isopropyl acetate. The solution was treated with heptanes (20 L) and stirred for 15 hours, at which time the concentration of product in the mother liquor was measured by HPLC at 11 mg/mL. The product was collected by filtration, rinsed with a solution of 1:1 (v/v) isopropyl acetate/heptanes (10 L), and dried under vacuum (100 mm Hg with a nitrogen sweep at 55° C.) to provide 5.89 kg (89% yield) of the desired product with a chiral purity of 99.8% ee. Chiral HPLC conditions: Daicel Chiral PAK AD 4.6×250 mm column at ambient temperature 0.3% v/v trifluoroacetic acid in ethanol (200 proof) over 30 minutes with a flow rate of 0.3 mL/min, UV detection at 243 nM. Retention times: desired product, ˜17 min; enantiomer, ˜14 min.

¹H NMR (300 MHz, CDCl₃) δ 8.40 (s, 1H), 7.85-7.90 (m, 0.5H), 7.80-7.90 (m, 2H), 7.20-7.35 (m, 2H), 7.05-7.15 (m, 4H), 4.75-4.85 (m, 0.5H), 4.20-4.35 (m, 2H), 4.0-4.15 (m, 1H), 3.75-3.90 (m, 2H), 3.35 (dd, 0.5H), 3.10 (dd, 0.5H), 1.42 (s, 3H), 1.30 (s, 3H); two rotomers of the formamide are observed for some signals.

### EXAMPLE 7

### 1-bromo-4-(methylsulfonyl)benzene

A 3L flask equipped with a mechanical stirrer, a thermocouple, an addition funnel, and a nitrogen inlet was placed in a warm water bath (35° C.), charged with 4-bromothioanisole (60 g, 0.295 mol), and ethanol (120 mL), and stirred until the solids dissolved. The water bath was replaced with a cooling bath and the mixture was allowed to cool to ambient temperature. The mixture was treated with a solution of OXONE® (potassium peroxymonosulfate, 240 g) in water (1200 mL) over 1 hour at such a rate as to keep the internal temperature <55° C. and stirred until HPLC showed <1% of the sulfoxide intermediate remained (about 5 hours). HPLC conditions: Zorbax Rx-C8 4.6 mm×25 cm column; mobile phase was a gradient of 70% water with 0.1% H₃PO₄/30% acetonitrile to 30% water with 0.1% phosphoric acid/70% acetonitrile over 15 minutes and at a flow rate of 1.5 mL/min followed by a 5 minute hold at 30/70; UV detection at 220 nM. Retention times: starting material, 10.9 min; sulfoxide intermediate, 4.9 min; product, 6.1 min.

The mixture was diluted with 5% wt sodium bicarbonate solution (600 mL), and stirred for 30 minutes. The precipitate was filtered, washed with water (1 L), and dried at 50° C. under vacuum (100 mm Hg with nitrogen bleed) until HPLC showed greater than 97% weight to provide 65.69 g (94.6%) of the desired product.

¹H NMR (300 MHz, CDCl₃) δ 7.82 (d, 2H), 7.72 (d, 2H), 3.07 (s, 3H).

### EXAMPLE 8

### 1-(methylsulfonyl)-4-[4′-(trifluoromethoxy)phenoxy]benzene

A reaction vessel equipped with a mechanical stirrer, reflux condenser, a thermocouple, and a nitrogen inlet was charged with potassium phosphate (114.0 g, 0.537 mol), Example 7 (60 g, 0.295 mol), 4-(trifluoromethoxy)phenol (47.8 g, 0.268 mol), and DMF (96 mL). The mixture was heated to 130° C. and stirred until HPLC showed <0.5% area 4-(trifluoromethoxy)phenol (about 13 hours). HPLC conditions: Zorbax Rx-C8 4.6 mm×25 cm column; mobile phase was a gradient of 70% water with 0.1% H₃PO₄/30% acetonitrile to 30% water with 0.1% phosphoric acid/70% acetonitrile over 15 minutes at a flow rate of 1.5 mL/min followed by a 5 minute hold at 30/70; UV detection at 220 nM. Retention times: starting sulfone, 6.15 min; starting phenol, 7.8 min; product, 10.7 min.

The mixture was cooled to room temperature, diluted with water (240 mL), and extracted with toluene (600 mL and 120 mL). The toluene layer was washed with 1 N NaOH solution (300 mL) and water (2×300 mL), filtered, concentrated to about 120 g, and azeotropically distilled with toluene (120 mL). The concentrate was treated slowly with heptane (900 mL) with agitation, stirred for 2 hours, and cooled to 0-5° C. until the mother liquor had a product concentration <5 mg/mL. The precipitate was collected by filtration, washed with heptane (120 mL), and dried under vacuum (100 mm Hg with nitrogen sweep) at 40° C. to provide 81.44 g (88.1%) of the desired product. The crude product can be further purified by recrystallization from toluene/heptane (90 mL/900 mL).

¹H NMR (300 MHz, CDCl₃) δ 7.94-7.89 (d, 2H), 7.31-7.25 (d, 2H), 7.13-7.07 (d, 4H), 3.06 (s, 3H).

What is claimed is:

1. A process for preparing a compound of formula (8) 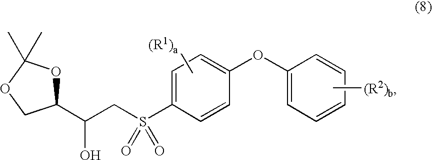
or a therapeutically acceptable salt thereof, wherein
a is 0, 1, or 2;
b is 0, 1, 2, or 3;
each R¹ is independently selected from the group consisting of alkyl, halo, nitro, and perfluoroalkyl; and
each R² is independently selected from the group consisting of alkoxy, alkyl, perfluoroalkoxy, and perfluoroalkyl;
the process comprising:
(a) reacting a compound of formula (3) 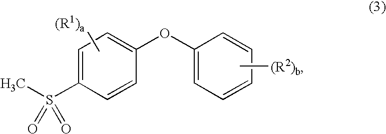
with a mixture of n-butyllithium and lithium hexamethyldisilazide;
(b) reacting the product of step (a) with a compound of formula (6) 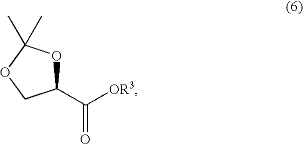
wherein R³ is alkyl; and
(c) reacting the product of step (b) with a reducing agent.

2. The process of claim 1 wherein
a is 0;
b is 1; and
R² is perfluoroalkoxy.

3. The process of claim 1 wherein step (a) is conducted in a solvent selected from the group consisting of tetrahydrofuran, diethyl ether, methyl tert-butyl ether, hexanes, toluene, tetramethylethylenediamine, and mixtures thereof.

4. The process of claim 3 wherein the solvent is a mixture of tetrahydrofuran and hexanes.

5. The process of claim 1 wherein step (a) is conducted at about −78° C. to about −40° C. for about 1 to about 6 hours.

6. The process of claim 1 wherein step (b) is conducted in a solvent selected from the group consisting of tetrahydrofuran, diethyl ether, methyl tert-butyl ether, hexanes, toluene, and mixtures thereof.

7. The process of claim 6 wherein the solvent is a mixture of tetrahydrofuran and hexanes.

8. The process of claim 1 wherein step (b) is conducted at about −78° C. to about −40° C. for about 30 minutes to about 6 hours.

9. The process of claim 1 wherein the reducing agent is selected from the group consisting of sodium borohydride, sodium triacetoxyborohydride, sodium cyanoborohydride, and lithium borohydride.

10. The process of claim 9 wherein the reducing agent is sodium borohydride.

11. The process of claim 1 wherein step (c) is conducted in a solvent selected from the group consisting of ethanol, methanol, tetrahydrofuran, diethyl ether, toluene, and mixtures thereof.

12. The process of claim 11 wherein the solvent is a mixture of ethanol and tetrahydrofuran.

13. The process of claim 1 wherein step (c) is conducted at about −20° C. to about 5° C. for about 30 minutes to about 12 hours.

14. The process of claim 1 which is a continuous process.

15. A process for preparing a compound of formula (10) 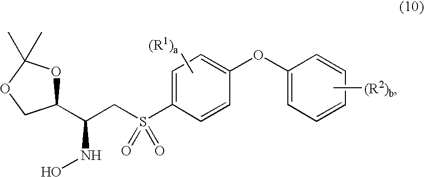
or a therapeutically acceptable salt thereof, wherein
a, b, R¹, and R² are as described in claim 1;
the process comprising:
(a) reacting a compound of formula (8) with methanesulfonyl chloride in the presence of a base; and
(b) reacting a solution of the product of step (a) in methyl tert-butyl ether with N-hydroxylamine.

16. The process of claim 15 wherein
a is 0;
b is 1; and
R² is perfluoroalkoxy.

17. The process of claim 15 wherein the compound of formula (10) is (4S)-4-[(1S)-1-(hydroxyamino)-2-({4-[4′-(trifluoromethoxy)phenoxy]phenyl }sulfonyl)ethyl]-2,2-dimethyl-1,3-dioxolane.

18. The process of claim 15 wherein the base is selected from the group consisting of triethylamine, 1,8-diazabicyclo[4.3.0]undec-7-ene, pyridine, 2,6-lutidine, 1-methylimidazole, 4-dimethylaminopyridine, and diisopropylethylamine.

19. The process of claim 18 wherein the base is triethylamine.

20. The process of claim 15 wherein step (a) is conducted in a solvent selected from the group consisting of ethyl acetate, isopropyl acetate, tetrahydrofuran, diethyl ether, toluene, and mixtures thereof.

21. The process of claim 20 wherein the solvent is ethyl acetate.

22. The process of claim 15 wherein step (a) is conducted at about −10° C. to about 30° C. for about 1 to about 8 hours.

23. The process of claim 15 wherein step (b) is conducted at about −20° C. to about 0° C. for about 4 to about 24 hours.

24. The process of claim 15 which is a continuous process.

25. A process for preparing a compound of formula (11a) 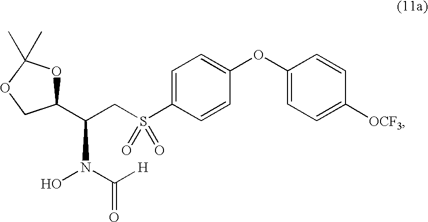
the process comprising:
(a) reacting a compound of formula (1a) 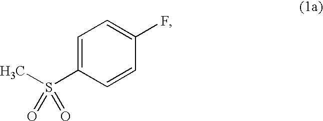
with a compound of formula (2a) 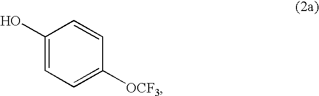
in the presence of a base;
(b) reacting the product of step (a) with a mixture of n-butyllithium and lithium hexamethyldisilazide;
(c) reacting the product of step (b) with a compound of formula (6a) 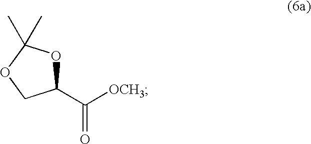
(d) reacting the product of step (c) with a reducing agent;
(e) reacting the product of step (d) with methanesulfonyl chloride in the presence of a base;
(f) reacting a solution of the product of step (e) in methyl tert-butyl ether with N-hydroxylamine; and
(g) reacting the product of step (f) with a formylating agent.

26. A process for preparing a compound of formula (11a),
the process comprising:
(a) reacting a compound of formula (1a) with a compound of formula (2a) in the presence of potassium hydroxide in dimethyl sulfoxide at about 75° C. to about 100° C. for about 8 to about 24 hours;
(b) reacting the product of step (a) with a mixture of n-butyllithium and lithium hexamethyldisilazide in a mixture of tetrahydrofuran and hexanes at about −78° C. to about −40° C. for about 1 to about 6 hours;
(c) reacting the product of step (b) with a compound of formula (6a) in a mixture of tetrahydrofuran and hexanes at about −78° C. to about −40° C. for about 30 minutes to about 6 hours;
(d) reacting the product of step (c) with sodium borohydride in a mixture of ethanol and tetrahydrofuran at about −20° C. to about 5° C. for about 30 minutes to about 12 hours;
(e) reacting the product of step (d) with methanesulfonyl chloride in the presence of triethylamine in ethyl acetate at about −10° C. to about 30° C. for about 1 to about 8 hours;
(f) reacting a solution of the product of step (e) in methyl tert-butyl ether with N-hydroxylamine at about −20° C. to about 0° C. for about 4 to about 24 hours; and
(g) reacting the product of step (f) with 2,2,2-trifluoroethyl formate and formic acid in buffered isopropyl acetate at about 45° C. to about 75° C. for about 1 to about 10 hours.

27. A process for preparing a compound of formula (11a), the process comprising:
(a) reacting a compound of formula (4a) 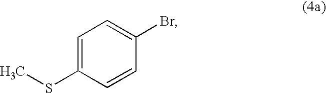
with an oxidizing agent;
(b) reacting the product of step (a) with a compound of formula (2a) in the presence of a base;
(c) reacting the product of step (b) with a mixture of n-butyllithium and lithium hexamethyldisilazide;
(d) reacting the product of step (c) with a compound of formula (6a);
(e) reacting the product of step (d) with a reducing agent;
(f) reacting the product of step (e) with methanesulfonyl chloride in the presence of a base;
(g) reacting a solution of the product of step (f) in methyl tert-butyl ether with N-hydroxylamine; and
(h) reacting the product of step (g) with a formylating agent.

28. A process for preparing a compound of formula (11a),
the process comprising:
(a) reacting a compound of formula (4a) with potassium peroxymonosulfate in a mixture of ethanol and water at about 20° C. to about 45° C. for about 1 to about 10 hours;
(b) reacting the product of step (b) with a compound of formula (2a) in the presence of potassium phosphate in N,N-dimethylformamide at about 100° C. to about 140° C. for about 8 to about 20 hours;
(c) reacting the product of step (b) with a mixture of n-butyllithium and lithium hexamethyldisilazide in a mixture of tetrahydrofuran and hexanes at about −78° C. to about −40° C. for about 1 to about 6 hours;
(d) reacting the product of step (c) with a compound of formula (6a); in a mixture of tetrahydrofuran and hexanes at about −78° C. to about −40° C. for about 30 minutes to about 6 hours;
(e) reacting the product of step (d) with sodium borohydride in a mixture of ethanol and tetrahydrofuran at about −20° C. to about 5° C. for about 30 minutes to about 12 hours;
(f) reacting the product of step (e) with methanesulfonyl chloride in the presence of triethylamine in ethyl acetate at about −10° C. to about 30° C. for about 1 to about 8 hours;
(g) reacting a solution of the product of step (f) in methyl tert-butyl ether with N-hydroxylamine at about −20° C. to about 0° C. for about 4 to about 24 hours; and
(h) reacting the product of step (g) with 2,2,2-trifluoroethyl formate and formic acid in buffered isopropyl acetate at about 45° C. to about 75° C. for about 1 to about 10 hours.
